(19) United States
(12) Patent Application Publication
(10) Pub. No.: US 2023/0295010 A1
(43) Pub. Date: Sep. 21, 2023

(54) Method for Recycling Selenium Source in Selenium-Enriched Yeast Fermentation Process, and Treatment System for Selenium-Containing Wastewater

(71) Applicant: ANGEL YEAST CO., LTD., Yichang, Hubei (CN)

(72) Inventors: Xiuji LIU, Yichang, Hubei (CN); Tao XIONG, Yichang, Hubei (CN); Xiao LI, Yichang, Hubei (CN); Weili DU, Yichang, Hubei (CN); Ming ZHAI, Yichang, Hubei (CN)

(21) Appl. No.: 18/016,671

(22) PCT Filed: Jul. 30, 2021
(86) PCT No.: PCT/CN2021/109841

§ 371 Date: Jan. 17, 2023

(30) Foreign Application Priority Data
Aug. 5, 2020 (CN) 202010779325.X

(51) Int. Cl. C02F 1/04; C12N 1/16; C02F 1/38

(52) U.S. Cl. CPC C02F 1/046; C12N 1/16; C02F 1/38; C02F 2103/32

(57) ABSTRACT

The present application provides a method for recycling selenium sources in a selenium-enriched yeast fermentation process, and a treatment system for selenium-containing wastewater. The method includes: step S1, performing separation and concentration treatments on selenium-containing wastewater produced in a selenium-enriched yeast fermentation process, so as to obtain a selenium-containing concentrated solution; step S2, adjusting a selenium content of the selenium-containing concentrated solution to a predetermined selenium content, so as to obtain a selenium-containing nutrient solution; and step S3, adding the selenium-containing nutrient solution into a selenium-enriched yeast fermentation reaction, so as to realize the recycling of the selenium source.

### TECHNICAL FIELD

The present application relates to the field of selenium-enriched yeast fermentation, in particular to a method for recycling a selenium source in a selenium-enriched yeast fermentation process, and a treatment system for selenium-containing wastewater.

### BACKGROUND

A production process of a selenium-enriched yeast is to add a selenium source into a yeast fermentation medium. The yeast uses its own metabolism to absorb the selenium source and convert the same into intracellular organic selenium, which can be better absorbed and utilized by human bodies, animals and plants.

Under normal circumstances, the conversion rate of selenium in a yeast fermentation process can only reach 30 to 70%, and the remaining selenium source that has not been absorbed is mainly left in fermentation liquor and is directly treated as fermentation wastewater, which not only results in waste of a large amount of selenium resources, but also increases the cost of subsequent environmental protection treatments and the risk of environmental pollution.

In the existing selenium-enriched fermentation wastewater treatment technology, which is mainly introduced in the CN103101987 patent “a device and method for utilizing selenium-enriched yeast wastewater” that, the wastewater is first evaporated and concentrated, and then is spray-dried to make dry powder, which is used as a fertilizer or feed. However, the environmental protection treatment cost of this technology is relatively high, and the value of the selenium source has not been fully utilized.

Therefore, in the face of the existing problems of waste of selenium resources in the selenium-enriched yeast fermentation process, and increases in subsequent environmental protection risks and costs, it is urgent to find an effective solution.

### SUMMARY

One of the main purposes of the present application is to provide a method for recycling a selenium source in a selenium-enriched yeast fermentation process, and a treatment system for selenium-containing wastewater, so as to solve the above problems of waste of the selenium resource in the selenium-enriched yeast fermentation process, and increases in subsequent environmental protection risks and costs.

A method for recycling a selenium source in a selenium-enriched yeast fermentation process provided by the present application includes: step S1, performing separation and concentration treatments on selenium-containing wastewater produced in a selenium-enriched yeast fermentation process, so as to obtain a selenium-containing concentrated solution; step S2, adjusting a selenium content of the selenium-containing concentrated solution to a predetermined selenium content, so as to obtain a selenium-containing nutrient solution; and step S3, adding the selenium-containing nutrient solution into a selenium-enriched yeast fermentation reaction, so as to realize the recycling of the selenium source.

The step S1 can further include: step S11, performing centrifugal separation on fermentation liquor produced in the selenium-enriched yeast fermentation process, so as to obtain a heavy phase and a light phase; step S12, collecting the light phase, and performing a concentration treatment on the light phase; and step S13, performing solid-liquid separation on the concentrated light phase, and removing solid substances to obtain the selenium-containing concentrated solution.

Further, the concentration treatment mentioned in the step S12 is a reduction vaporization concentration treatment.

Further, the solid-liquid separation in the step S13 is centrifuge separation or membrane filtering separation.

Further, the step S2 includes: adding an inorganic selenium source and/or an organic selenium source into the selenium-containing concentrated solution.

Preferably, in the step S1, the selenium content of the selenium-containing concentrated solution after the centrifugal treatment is more than 500 ppm.

Further, in the method for recycling the selenium source provided by the present application, the steps S1 to S3 are circulated for at least twice, so that the selenium source is fully utilized, and the utilization rate is increased by at least 30%.

Another aspect of the present application provides a treatment system for selenium-containing wastewater. The treatment system includes: a first solid-liquid separation device, including a first inlet and a first separation liquid outlet; a concentration device, including a second inlet and a concentrated solution outlet, wherein the second inlet is connected to the first separation liquid outlet; a second solid-liquid separation device, including a concentrated solution inlet and a second separation liquid outlet, wherein the concentrated solution inlet is connected to the concentrated solution outlet; and a selenium adjusting device, including a third inlet, a selenium source inlet and a selenium-containing nutrient solution outlet, wherein the third inlet is connected to the second separation liquid outlet.

Further, the first solid-liquid separation device is a centrifuge.

Further, the second solid-liquid separation device is a centrifuge or a membrane filtering device.

Further, the treatment system further includes a selenium-containing nutrient solution conveying device, which is connected to the selenium adjusting device and conveys the selenium-containing nutrient solution into a fermentation reaction tank.

Further, the selenium adjusting device in the treatment system further includes a selenium content testing device.

By applying the technical solutions of the present application, a method and system for recycling a selenium source in a selenium-enriched yeast fermentation process are provided for the first time, that is, after a bath of selenium-enriched yeast fermentation, the selenium source in the selenium-containing wastewater is recycled, and is reused in the next batch of selenium-enriched yeast fermentation, thereby improving the utilization rate of the selenium source. At the same time, zero discharge of selenium wastewater is basically realized, and thus the method and system can be directly applied to industrial production.

### BRIEF DESCRIPTION OF THE DRAWINGS

The drawings forming a part of the present application are used for providing a further understanding of the present application, and exemplary embodiments of the present application and descriptions thereof are used for explaining the present application, but do not constitute improper limitations to the present application. In the drawings:

FIG. 1 shows a schematic flow diagram of a method for recycling a selenium source in a selenium-enriched yeast fermentation process provided according to an embodiment of the present application;

FIG. 2 shows a schematic flow diagram of step S1 in the method for recycling the selenium source shown in FIG. 1; and

FIG. 3 shows a schematic diagram of a connection relationship of a treatment system for selenium-containing wastewater provided according to an embodiment of the present application.

The above-mentioned drawings include the following reference signs:

10. first solid-liquid separation device; 20. concentration device; 30. second solid-liquid separation device; 40. selenium adjusting device; 50. selenium-containing nutrient solution conveying device.

### DETAILED DESCRIPTION OF THE EMBODIMENTS

It should be noted that, embodiments in the present application and features in the embodiments can be combined with each other under the condition of no conflict. The present application will be described in detail below in combination with the embodiments.

### Description of Terms

A “predetermined selenium content” mentioned in the present application refers to a selenium content suitable for a yeast fermentation process, which is used by those skilled in the art according to the type of an added selenium source and the conditions of a yeast in a selenium-enriched yeast fermentation process.

“Selenium-enriched yeast fermentation” mentioned in the present application includes, but is not limited to, hydrolytic sugar of the starch, molasses or a mixture of hydrolytic sugar of the starch and molasses.

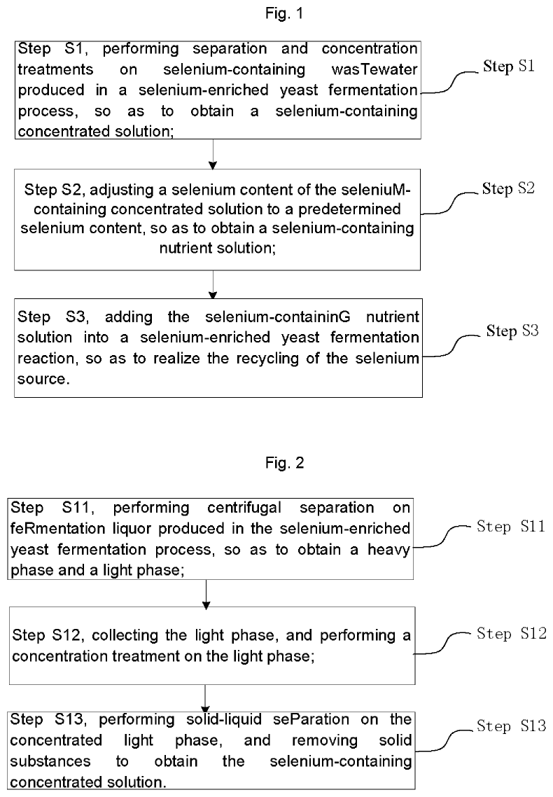

As is well-known, in the selenium-enriched yeast fermentation process, due to a limited selenium conversion rate of the yeast, selenium-containing wastewater contains a large amount of unused selenium sources. In addition, the selenium-containing wastewater also contains a large amount of sugars, proteins, amino acids, pigments, pectin and other substances, thus is high-concentration organic wastewater that is difficult to deal with, and serious environmental pollution will be caused if the selenium-containing wastewater is directly discharged. In order to solve this problem, the present application provides a method for recycling a selenium source in a selenium-enriched yeast fermentation process, which not only solves the problem of waste of the selenium source, but also realizes an environmental beneficial effect of zero discharge of selenium wastewater. The method for recycling the selenium source provided by the present application, as shown in FIG. 1, includes: step S1, performing separation and concentration treatments on selenium-containing wastewater produced in a selenium-enriched yeast fermentation process, so as to obtain a selenium-containing concentrated solution; step S2, adjusting a selenium content of the selenium-containing concentrated solution to a predetermined selenium content, so as to obtain a selenium-containing nutrient solution; and step S3, adding the selenium-containing nutrient solution into a selenium-enriched yeast fermentation reaction, so as to realize the recycling of the selenium source.

By means of the above method, not only the selenium source in the wastewater is recovered, but the selenium source is also recycled in the selenium-enriched yeast fermentation process, thereby reducing the waste of the selenium source. Moreover, by adjusting the selenium content in the selenium-containing concentrated solution to a predetermined concentration, not only the selenium content requirement in the yeast fermentation process is ensured, but the amount of selenium source added in the next batch of fermentation process is also reduced. By means of the continuous circulation of the above steps of the method, zero discharge of selenium wastewater is truly realized.

In an embodiment provided by the present application, as shown in FIG. 2, the step S1 can further include: step S11, performing centrifugal separation on fermentation liquor produced in the selenium-enriched yeast fermentation process, so as to obtain a heavy phase and a light phase; step S12, collecting the light phase, and performing a concentration treatment on the light phase; and step S13, performing solid-liquid separation on the concentrated light phase, and removing solid substances to obtain the selenium-containing concentrated solution. Preferably, in the step S13, the selenium content of the selenium-containing concentrated solution after the centrifugal treatment is more than 500 ppm. If the selenium content in the selenium-containing concentrated solution is greater than this numerical value, both a dissolution volume and a final fermentation volume of the selenium source can be optimized, and the fermentation process can be controlled more easily. Supernate obtained after the centrifugal treatment in the above step S13 is the selenium-containing concentrated solution, which is entirely added into the next batch of fermentation. By continuously circulating the above procedures, zero discharge of selenium-containing wastewater can be realized.

In an embodiment provided by the present application, the concentration treatment mentioned in the step S12 is a reduction evaporation concentration process. Compared with a conventional membrane concentration process in the prior art, the reduction evaporation concentration process is simpler, but it is not limited to the above concentration treatment manner, and can also be membrane concentration, ion exchange, electrodialysis and other manners, and methods capable of concentrating the selenium source in the solution all fall within the protection scope of the present application. Further, the solid-liquid separation in the step S13 can be centrifuge separation or membrane filtering separation, and methods capable of performing solid-liquid separation on the solution all fall within the protection scope of the present application.

In an embodiment provided by the present application, the step S2 is to add an inorganic selenium source and/or an organic selenium source into the selenium-containing concentrated solution, wherein the inorganic selenium source can be common inorganic selenium sources such as sodium selenite, sodium selenate, zinc selenite or selenium dioxide, and the organic selenium source can include selenium-containing amino acids, selenium-containing polypeptides, selenium-containing proteins, selenium-containing polysaccharides, etc. One or more of the inorganic selenium source and the organic selenium source can be added into the selenium-containing concentrated solution simultaneously or separately as the selenium source.

Further, in the method for recycling the selenium source provided by the present application, the steps S1 to S3 are circulated for at least twice, so that the selenium source is fully utilized, and the utilization rate is increased by at least 30%.

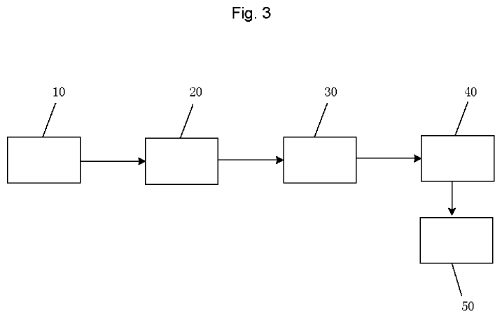

Another purpose of the present application is to provide a treatment system for selenium-containing wastewater. As shown in FIG. 3, the treatment system includes: a first solid-liquid separation device 10, including a first inlet and a first separation liquid outlet; a concentration device 20, including a second inlet and a concentrated solution outlet, wherein the second inlet is connected to the first separation liquid outlet; a second solid-liquid separation device 30, including a concentrated solution inlet and a second separation liquid outlet, wherein the concentrated solution inlet is connected to the concentrated solution outlet; and a selenium adjusting device 40, including a third inlet, a selenium source inlet and a selenium-containing nutrient solution outlet, wherein the third inlet is connected to the second separation liquid outlet.

Further, the second solid-liquid separation device 30 is a centrifuge or a membrane filtering device.

Further, the treatment system further includes a selenium-containing nutrient solution conveying device 50, which is connected to the selenium adjusting device 40 and conveys the selenium-containing nutrient solution into a fermentation reaction tank.

Further, the selenium adjusting device 40 in the treatment system further includes a selenium content testing device.

By using the treatment system, selenium-containing wastewater can be directly used as the selenium-containing nutrient solution in the fermentation reaction process, so that the selenium source is recycled in the fermentation process, and zero discharge of selenium wastewater is realized.

### Embodiment 1

A selenium-enriched yeast was fermented by using a molasses process, and sodium selenite was used as a selenium source to perform a 50 L small-scale fermentation experiment.

The total amount of sodium selenite added in a first batch of selenium-enriched yeast fermentation was 20 g, the tank storage volume was 31 L at the end of the fermentation, fermentation liquor was separated by a centrifuge, the rotation speed was controlled at 5000 rpm, a heavy phase was collected as the selenium-enriched yeast, which was 1.55 kg in total by being dried, and the selenium content was 3134.2 ppm. A total of 24 L of light phase was collected and was transferred to a 50 L rotary evaporator, the temperature was controlled at 80 to 90 DEG C for evaporation and concentration after vacuumizing, after the concentration was ended, centrifugation was performed by using the centrifuge at a rotation speed of 5000 rpm, solid wastes were removed to obtain 3. 2L of high-concentration selenium-containing concentrated solution, and it was detected that the selenium content of the selenium-containing concentrated solution was 884.9 ppm.

It was calculated that the high-concentration selenium-containing concentrated solution contained 6.2 g of sodium selenite, 13.8 g of sodium selenite was directly added into the selenium-containing concentrated solution, to reach 20 g of sodium selenite in total, then obtain selenium-containing nutrient solution. Control was performed with reference to the first batch of fermentation, the frequency of selenium addition and selenium addition amount in the fermentation process were kept consistent with those in the first batch of fermentation, the selenium addition structure, the selenium addition amount and the selenium addition type were also kept consistent, and the selenium-containing nutrient solution was added into a second batch of selenium-enriched yeast fermentation.

At the end of the fermentation, the tank storage volume was 35.6 L, the fermentation liquor was separated by the centrifuge, the rotation speed is controlled at 5000 rpm, the heavy phase was collected as the selenium-enriched yeast, which was 1.74 kg in total by being dried, and the selenium content was 3069.9 ppm. A total of 27 L of light phase was collected and was transferred to the 50 L rotary evaporator, the temperature was controlled at 80-90 DEG C for evaporation and concentration after vacuumizing, after the concentration was ended, centrifugation was performed by using the centrifuge at the rotation speed of 5000 rpm to obtain 3.0 L of high-concentration selenium-containing concentrated solution, and it was detected that the selenium content of the concentrated solution is 989.2 ppm.

The same as above, it was calculated that the selenium-containing concentrated solution contained 6.5 g of sodium selenite, 13.5 g of sodium selenite was directly added into the selenium-containing concentrated solution to reach 20 g of sodium selenite in total, then obtain selenium-containing nutrient solution. Control was performed with reference to the first batch of fermentation, the frequency of selenium addition and the selenium addition amount in the fermentation process were kept consistent with those in the first batch of fermentation, and the selenium-containing nutrient solution was added into a third batch of selenium-enriched yeast fermentation. At the end of the fermentation, the tank storage volume was 35.3 L, the fermentation liquor was separated by the centrifuge, the rotation speed was controlled at 5000 rpm, the heavy phase was collected as the selenium-enriched yeast, which was 1.67 kg in total by being dried, and the selenium content was 3022.3 ppm.

After shown by calculation, the utilization rate of the first batch of fermented selenium was 53.4%, after the selenium source was recycled to replace part of sodium selenite, two consecutive batches of fermentation were performed, and counted by the amount of newly added sodium selenite, the comprehensive utilization rate of selenium reached 84.9% and 81.7%, respectively.

TABLE 1
Calculation of selenium utilization rate in Embodiment 1

| | Tank | Sodium | | Selenium | Selenium |
|---|---|---|---|---|---|
| Fermentation | storage | selenite/ | Yield of | content/ | utilization |
| batch | volume/L | g | yeast/g | ppm | rate/% |
| First batch of | 31.0 | 20 | 1557.9 | 3134.2 | 53.4 |
| fermentation | | | | | |
| Second batch | 35.6 | 13.8 | 1744.0 | 3069.9 | 84.9 |
| of fermentation | | | | | |
| Third batch of | 35.3 | 13.5 | 1668.0 | 3022.3 | 81.7 |
| fermentation | | | | | |

### Embodiment 2

A selenium-enriched yeast was fermented by using an amylohydrolysis sugar process, and sodium selenite was used as a selenium source to perform a 50 L small-scale fermentation experiment.

The total amount of sodium selenite added in a first batch of selenium-enriched yeast fermentation was 17 g, the tank storage volume was 30.3 L at the end of the fermentation, fermentation liquor was separated by a centrifuge, the rotation speed was controlled at 5000 rpm, a heavy phase was collected as the selenium-enriched yeast, which was 1.41 kg in total by being dried, and the selenium content was 3526.1 ppm. A total of 23.4 L of light phase was collected and was transferred to a 50 L rotary evaporator, the temperature was controlled at 80 to 90 DEG C for evaporation and concentration after vacuumizing, after the concentration was ended, filtration was performed by using a 0.45-micron filter membrane, solid wastes were removed to obtain 3.5 L of selenium-containing concentrated solution, and it was detected that the selenium content was 821.8 ppm.

It was calculated that the selenium-containing concentrated solution contained 6.3 g of sodium selenite, 10.7 g of sodium selenite was directly added into the selenium-containing concentrated solution, to reach 17 g of sodium selenite in total, then obtain selenium-containing nutrient solution. Control was performed with reference to the first batch of fermentation, the frequency of selenium addition and selenium addition amount in the fermentation process were kept consistent with those in the first batch of fermentation, and the selenium-containing nutrient solution was added into a second batch of selenium-enriched yeast fermentation.

At the end of the fermentation, the tank storage volume was 34.4 L, the fermentation liquor was separated by the centrifuge, the rotation speed was controlled at 5000 rpm, the heavy phase was collected as the selenium-enriched yeast, which was 1.52 kg in total by being dried, and the selenium content was 3321.9 ppm. A total of 26 L of light phase was collected and was transferred to the 50 L rotary evaporator, the temperature was controlled at 80 to 90 DEG C for evaporation and concentration after vacuumizing, after the concentration was ended, the material volume was 3.3 L, filtration was performed by using the 0.45-micron filter membrane, solid wastes were removed to obtain a high-concentration selenium-containing concentrated solution, and it was detected that the selenium content of the concentrated solution was 802.5 ppm.

The same as above, it was calculated that the selenium-containing concentrated solution contained 5.8 g of sodium selenite, 11.2 g of sodium selenite was directly added into the concentrated solution, to reach 17 g of sodium selenite in total, then obtain selenium-containing nutrient solution. Control was performed with reference to the first batch of fermentation, the frequency of selenium addition and the selenium addition amount in the fermentation process were kept consistent with those in the first batch of fermentation, and the selenium-containing nutrient solution was added into a third batch of selenium-enriched yeast fermentation. At the end of the fermentation, the tank storage volume was 34.9 L, the fermentation liquor was separated by the centrifuge, the rotation speed was controlled at 5000 rpm, the heavy phase was collected as the selenium-enriched yeast, which was 1.44 kg in total by being dried, and the selenium content was 3355.7 ppm.

After shown by calculation, the utilization rate of the first batch of fermented selenium was 64.1%, after the selenium source was recycled to replace part of sodium selenite, two consecutive batches of fermentation were performed, and counted by the amount of newly added sodium selenite, the comprehensive utilization rate of selenium reaches 96.7% and 94.5%%, respectively.

TABLE 2
Calculation of selenium utilization rate in Embodiment 2

| | Tank | Sodium | | Selenium | Selenium |
|---|---|---|---|---|---|
| Fermentation | storage | selenite/ | Yield of | content/ | utilization |
| batch | volume/L | g | yeast/g | ppm | rate/% |
| First batch of | 30.3 | 17 | 1411.8 | 3526.1 | 64.1 |
| fermentation | | | | | |
| Second batch | 34.4 | 10.7 | 1523.7 | 3321.9 | 96.7 |
| of fermentation | | | | | |
| Third batch of | 34.9 | 11.2 | 1442.1 | 3355.7 | 94.5 |
| fermentation | | | | | |

The specific embodiments of the present application have been described above through embodiments. Those skilled in the art should understand that, the above embodiments are only for the purpose of examples, and should not be considered as limiting the protection scope of the present application. Those skilled in the art can make modifications, changes or replacements without departing from the spirits of the present application, but various equivalent changes made according to the present application still belong to the scope covered by the present application.

1. A method for recycling a selenium source in a selenium-enriched yeast fermentation process, wherein the method comprises:
step S1, performing separation and concentration treatments on selenium-containing wastewater produced in a selenium-enriched yeast fermentation process, so as to obtain a selenium-containing concentrated solution;
step S2, adjusting a selenium content of the selenium-containing concentrated solution to a predetermined selenium content, so as to obtain a selenium-containing nutrient solution; and
step S3, adding the selenium-containing nutrient solution into a selenium-enriched yeast fermentation reaction, so as to realize the recycling of the selenium source.

2. The method according to claim 1, wherein the step S1 comprises:
step S11, performing centrifugal separation on fermentation liquor produced in the selenium-enriched yeast fermentation process, so as to obtain a heavy phase and a light phase;
step S12, collecting the light phase, and performing a concentration treatment on the light phase; and
step S13, performing solid-liquid separation on the concentrated light phase, and removing solid substances to obtain the selenium-containing concentrated solution.

3. The method according to claim 2, wherein the concentration treatment in the step S12 is a reduction vaporization concentration treatment.

4. The method according to claim 2, wherein the solid-liquid separation in the step S13 is centrifuge separation or membrane filtering separation.

5. The method according to claim 1, wherein the step S2 comprises: adding an inorganic selenium source and/or an organic selenium source into the selenium-containing concentrated solution.

6. The method according to claim 1, wherein the selenium content of the selenium-containing concentrated solution is more than 500 ppm.

7. The method according to claim 1, wherein the steps S1 to S3 are circulated for at least twice.

8. A treatment system for selenium-containing wastewater, wherein the treatment system comprises:
a first solid-liquid separation device, comprising a first inlet and a first separation liquid outlet;
a concentration device, comprising a second inlet and a concentrated solution outlet, wherein the second inlet is connected to the first separation liquid outlet;
a second solid-liquid separation device, comprising a concentrated solution inlet and a second separation liquid outlet, wherein the concentrated solution inlet is connected to the concentrated solution outlet; and
a selenium adjusting device, comprising a third inlet, a selenium source inlet and a selenium-containing nutrient solution outlet, wherein the third inlet is connected to the second separation liquid outlet.

9. The treatment system according to claim 8, wherein the first solid-liquid separation device is a centrifuge.

10. The treatment system according to claim 8, wherein the second solid-liquid separation device is a centrifuge or a membrane filtering device.

11. The treatment system according to claim 8, wherein the treatment system further comprises a selenium-containing nutrient solution conveying device, which is connected to the selenium adjusting device and conveys the selenium-containing nutrient solution into a fermentation reaction tank.

12. The treatment system according to claim 8, wherein the selenium adjusting device further comprises a selenium content testing device.

13. The method according to claim 3, wherein the solid-liquid separation in the step S13 is centrifuge separation or membrane filtering separation.

14. The method according to claim 2, wherein the step S2 comprises: adding an inorganic selenium source and/or an organic selenium source into the selenium-containing concentrated solution.

15. The method according to claim 3, wherein the step S2 comprises: adding an inorganic selenium source and/or an organic selenium source into the selenium-containing concentrated solution.

16. The method according to claim 4, wherein the step S2 comprises: adding an inorganic selenium source and/or an organic selenium source into the selenium-containing concentrated solution.

17. The treatment system according to claim 9, wherein the treatment system further comprises a selenium-containing nutrient solution conveying device, which is connected to the selenium adjusting device and conveys the selenium-containing nutrient solution into a fermentation reaction tank.

18. The treatment system according to claim 10, wherein the treatment system further comprises a selenium-containing nutrient solution conveying device, which is connected to the selenium adjusting device and conveys the selenium-containing nutrient solution into a fermentation reaction tank.

19. The treatment system according to claim 9, wherein the selenium adjusting device further comprises a selenium content testing device.

20. The treatment system according to claim 10, wherein the selenium adjusting device further comprises a selenium content testing device.
